(19) United States
(12) Patent Application Publication
(10) Pub. No.: US 2026/0157789 A1
(43) Pub. Date: Jun. 11, 2026

(54) FORCE TRANSMISSION ELEMENT, SURGICAL INSTRUMENT, AND METHOD FOR PRODUCING THE FORCE TRANSMISSION ELEMENT

(71) Applicant: KARL STORZ SE & Co. KG, Tuttlingen (DE)

(72) Inventors: Robin MERZ, Tuttlingen (DE); Benedikt HAHN, Straßberg (DE); Nico MOOSMANN, Tuttlingen (DE); Daniel KÄRCHER, Tuttlingen (DE)

(21) Appl. No.: 19/180,290

(22) Filed: Apr. 16, 2025

(30) Foreign Application Priority Data
Apr. 18, 2024 (DE) 10 2024 110 881.9

(51) Int. Cl. A61B 18/14; A61B 17/00; A61B 18/00

(52) U.S. Cl. CPC A61B 18/1442; A61B 2017/00367; A61B 2017/00526; A61B 2018/00077; A61B 2018/00083; A61B 2018/00595; A61B 2018/00601

(57) ABSTRACT

A force transmission element, in particular a pull rod for an endoscopic instrument, has: a potential-free rod configured for force transmission, which has at least one local clearance; and an electrode which extends over an outer surface of the clearance and in the axial direction along the clearance, wherein the electrode is in contact with an electrical conductor which extends within the rod. One end, a proximal end, of the rod has an electrically insulated positive-connection element which is configured to engage with a bearing element of a surgical instrument. Furthermore, the pull rod for an endoscopic instrument relates to a surgical instrument comprising such a force transmission element and to a method for producing such a force transmission element.

### CROSS REFERENCE TO RELATED APPLICATIONS

This application claims the benefit of priority under 35 U.S.C. § 119 of German Application 10 2024 110 881.9, filed Apr. 18, 2024, the entire contents of which are incorporated herein by reference.

### FIELD OF THE INVENTION

The present invention relates to a force transmission element for surgical instruments, in particular to a pull rod for an endoscopic instrument. Furthermore, the present invention relates to a surgical instrument comprising such a force transmission element and to a method for producing such a force transmission element.

### TECHNICAL BACKGROUND

Surgical instruments often have a mechanical mounting, in particular an anti-rotation device or an axial guide with a stop. In addition, surgical instruments often have electrical isolation (insulation). The mechanical mounting and electrical isolation are realized by separate components or elements of the surgical instrument.

For example, surgical tubular shaft instruments (e.g., endoscopes) are designed as reusable tubular shaft instruments. Depending upon the design of the instrument, in particular the shaft, the insulation shaft and the force transmission element can be completely separated from one another for cleaning the instrument. After cleaning, the instrument is reassembled and sterilized. Experience shows that the performance of sliding contacts, as provided on poles, diminishes due to cleaning—for example, due to oxidation. Therefore, sliding contacts must be minimized.

Furthermore, for example, bipolar operated surgical tubular shaft instruments (e.g., endoscopes with bipolar tools/accessories) have two electrical leads, which are usually formed by two components or elements of the surgical tubular shaft instrument that are electrically insulated from each other. In the case of bipolar surgical tubular shaft instruments, two electrodes (active electrode and neutral electrode) are arranged on the accessory of the surgical tubular shaft instrument. The high-frequency alternating current is introduced into the target tissue from a first electrode (active electrode) directly opposite the second electrode (neutral electrode).

### SUMMARY OF THE INVENTION

Against this background, the present invention is based upon the object of providing an improved medical instrument with a mechanical mounting and electrical isolation.

According to the invention, this object is achieved by a force transmission element having features according to the invention and/or by a surgical instrument having features according to the invention and/or by a method having features according to the invention.

Accordingly, according to a first aspect of the present invention, a force transmission element for surgical instruments, in particular a pull rod for an endoscopic instrument, is provided. The force transmission element comprises a rod and an electrode. The rod is potential-free, designed for force transmission, and has at least one local clearance. The electrode extends over an outer surface of the clearance and at least partially in the axial direction along the clearance, wherein the electrode is in contact with an electrical conductor extending within the rod. One end, in particular a proximal end, of the rod has an electrically insulated positive-connection element which is designed to engage with a bearing element or operating element of a surgical instrument.

Furthermore, according to a second aspect of the present invention, a surgical instrument is provided—in particular, a surgical tubular shaft instrument having a force transmission element according to the first aspect of the present invention, a tubular shaft, an actuation interface, a bearing element, and a tool. The force transmission element is housed in the tubular shaft. The actuation interface is designed to actuate the force transmission element. The bearing element is integrated into the actuation interface and couples the positive-connection element of the force transmission element. The tool can be moved using the force transmission element.

Furthermore, according to a third aspect of the present invention, a method for producing a force transmission element for surgical instruments, in particular a force transmission element according to the first aspect of the present invention, is provided, which comprises the following steps:
Providing a potential-free rod designed for force transmission, which has at least one local clearance and one end, in particular a proximal end, with an electrically insulated positive-connection element designed to engage with a bearing element of a surgical instrument.
Applying an electrode extending over an outer surface of the clearance and at least partially in the axial direction along the clearance, wherein the electrode is brought into contact with an electrical conductor extending within the rod.

The idea underlying the present invention is to transport current and a tensile/compressive force into the actuation interface via the force transmission element and/or pull rod, wherein the rod itself and the proximal end do not carry a current pole. The rod itself is not under electrical voltage.

The rod may be made at least partially of a metallic material such as steel or stainless steel. Furthermore, at least one potential-free, e.g., electrically insulating (electrically isolated), region or portion is provided. The rod can in particular be designed to transmit a force in the axial direction (tension or compression), and additionally or alternatively a torque. The axial force or torque can be transmitted from the actuation interface to the rod via the bearing element and passed through the rod to an accessory or tool of the surgical instrument in order to move the accessory or tool.

The clearance in the rod serves as an attachment region for the electrode. The clearance can be provided in a primary molding process or can be introduced into the rod by a machining process such as milling.

The function of current transmission is provided by the electrical conductor and the electrode. The electrical conductor extends in the axial direction in the rod and is electrically isolated from the environment by the rod. Optionally, the electrical conductor can have an electrically insulating sheath and/or coating. For example, the electrical conductor can be designed as a power cable. For certain applications, for example, a second electrode may be provided. The second electrode may extend over an outer surface of a second clearance and at least partially in the axial direction along the second clearance. In particular, in a bipolar operation of the surgical tool, the electric current can be conducted via the force transmission element by electrically connecting one of the two poles for conducting the electric current to the electrode and the other of the two poles for conducting the electric current to the second electrode. Both electrodes are each connected to their own electrical conductor, wherein the two electrical conductors are arranged electrically isolated from each other in the potential-free rod. This means that the rod insulates the two electrodes from each other. In particular, the rod insulates the electrode or the two electrodes from the positive-connection element. Consequently, the end and/or the positive-connection element is not in electrical contact with the electrical conductor or the electrode.

The force transmission element is inserted into the tubular shaft of the surgical instrument. The force transmission element is mounted or guided at least at the end, in particular the proximal end, of the rod in the surgical instrument. In particular, the rod is mounted or guided in the tubular shaft in such a way that the force transmission element can move or rotate axially relative to the tubular shaft. Thus, the force transmission element can transmit an axial force (tensile or compressive) and additionally or alternatively a torque within the tubular shaft and relative to it.

The actuation interface is used to actuate the force transmission element by applying an axial force or torque to the force transmission element. The bearing element of the actuation interface engages the positive-connection element of the force transmission element in an operational state. When the actuation interface is actuated, the bearing element transmits the axial force or torque to the positive-connection element of the rod and consequently actuates the force transmission element.

The tool, which may in particular be designed in the form of an interchangeable accessory, is moved by the axial force or torque transmitted via the force transmission element within and opposite the tubular shaft. The tool can be designed as a clamp, pliers, tweezers, gripper, scissors, and the like. In particular, the tool can also be designed to be operated monopolarly or bipolarly to, for example, cut tissue, cauterize it, and the like.

If the surgical instrument is to be operated by a user such as a surgeon, the surgical instrument includes a grip as a handle for the user. The actuation interface then transmits a force or torque, which is applied by the user to the handle via a suitable actuation (e.g., movable grip limb), to the force transmission element.

If the surgical instrument can be connected to a robot, the surgical instrument comprises a corresponding connection interface for the robot, which can apply an axial force or torque to the force transmission element via the actuation interface.

The force transmission element according to the invention with rod and electrode including electrical conductor provides a functional separation of mechanical force transmission and current transmission, since each component fulfills only one of the two functions. In addition, the force transmission element according to the invention can maintain a predetermined installation space and the required creepage distances between the electrode and the non-current-carrying components or parts, such as the positive-connection element of the rod. Furthermore, assembly effort can be significantly reduced.

Advantageous further developments and embodiments of the present invention are the subject matter of the corresponding dependent claims.

According to a further development of the present invention, the rod has a central portion and a contact portion, arranged between the central portion and the end, in which the at least one clearance is arranged, wherein the contact portion has a sleeve made of ceramic material. The sleeve can serve as a carrying frame for the other components. In particular, the sleeve is designed to be mechanically stable in such a way that the sleeve can be used as a load-bearing component without the use of other electrically insulating materials, such as plastic. According to one embodiment, the sleeve can, for example, be designed as an, in particular substantially, cylindrical body in which two or more bores are contained. The base of the cylinder shape can be circular, but other basic shapes are also conceivable. It can be a solid body or a hollow body. Additional recesses, modifications of shape, undercuts, or the like can be provided therein. In addition, the rod may have a flexible portion—for example, an elastically bendable or articulable section. Furthermore, the sleeve can be made of a glass material.

The clearance of the rod can, for example, be circumferential.

Optionally, the electrode can be directly integrated into the sleeve or contact portion using a 2-component process. This can further reduce assembly effort, reduce gaps, and/or reduce tolerance problems. In addition, a number of electrical transitions, such as soldering points, and/or connection points may be reduced. Furthermore, the sleeve can have an undercut, without the need to carry out any post-machining of the sleeve.

A ceramic material offers good mechanical stability and excellent electrical insulation properties.

According to a further development, the contact portion has a wire device for transmitting force between the central portion and the end, wherein the wire device extends in the axial direction along the rod and is at least partially surrounded by the sleeve. For example, the wire device is radially enclosed by the sleeve. The wire device is electrically insulated from the electrode and the electrical conductor by the ceramic material of the sleeve. Thus, the wire device can be made of a metal or comparable materials, for example, without being limited to electrically non-conductive materials.

The wire device preferably comprises two traction wires in order to transmit in particular a tensile force and optionally a bending moment between the end of the rod and the central portion. The two traction wires can each extend through the sleeve in separate bores.

According to a further development, the wire device is connected to the positive-connection element and the central portion of the rod with a material bond, wherein the sleeve is at least positively fastened by the wire device between the positive-connection element and the central portion. In addition, the sleeve may be glued at a contact point to the end and/or the central portion. For example, an end face of the sleeve may be glued to an end face of the end. Alternatively or additionally, the sleeve can be glued into the central portion of the rod.

According to a further development, the wire device is connected to the positive-connection element and the central portion with a material bond, in particular by means of a welded connection or by means of a capillary adhesive.

According to a further development, the sleeve is manufactured in one piece by a generative manufacturing process or by injection molding. In this way, subsequent machining of the sleeve, in particular a machining process, to create the clearance can be omitted. Using the generative manufacturing process or additive manufacturing, complex internal contours and a high aspect ratio of bore depth to bore diameter or total width to total length can be achieved. This allows a slimmer design to be realized, which in particular allows a diameter of the sleeve and the rod of less than about 2.5 mm.

According to a further development, the clearance has at least one substantially planar portion, parallel to the axial direction of the rod, which is located further inwards in the radial direction compared to an outer surface of the rod and contacts the electrode by lying against it, in particular lying flat against it.

According to a further development, the positive-connection element is spherical. The diameter of the spherical positive-connection element is at most one diameter of the rod. For example, the positive-connection element protrudes from the rod in the axial direction. The positive-connection element is made of metal, for example.

According to a further development, the tubular shaft is designed as a round tube. The rod of the force transmission element is designed as a round rod. The sleeve extends at least in sections circularly along the surface of the rod. The force transmission element is arranged concentrically in the tubular shaft.

The rod, designed as a round rod, has a substantially circular cross-section with a substantially constant diameter along the axial direction, except in the region of the clearance.

The tubular shaft, designed as a round tube, has an substantially circular cross-section with a substantially constant diameter along the axial direction. The force transmission element with a round rod is arranged concentrically in the tubular shaft designed as a round tube. The bearing element engages the positive-connection element with a positive connection in order to secure the force transmission element with the rod, designed as a round rod, in the tubular shaft designed as a round tube.

The bearing element of the surgical element engages the positive-connection element of the rod in such a way that axial displacement and, if necessary, rotation of the rod relative to the tubular shaft is possible.

In one embodiment, rotation of the rod relative to the tubular shaft may be blocked or limited by, for example, a longitudinal groove or a flattened region of the rod. This prevents unwanted rotation of the entire force transmission element relative to the tool or accessory of the surgical element, thus ensuring proper functioning of the tool/accessory.

The force transmission element and surgical instrument designed in this way are, advantageously, particularly easy to manufacture.

According to a further development of the present invention, the step of applying the electrode comprises stripping and welding, soldering, and/or crimping the electrical conductor to the electrode. If necessary, the electrical conductor can also be shortened to a suitable length. In particular, the stripped portion of the electrical conductor is welded to an inner side of the electrode.

According to a further development of the present invention, the rod has a central portion and a contact portion, arranged between the central portion and the end, in which the at least one clearance is arranged, wherein a wire device is pushed through a sleeve, made of ceramic material, provided in the contact portion. For example, the end is first mounted on the contact portion before the central portion is mounted on the contact portion.

According to a further development of the present invention, the method further comprises the step of materially bonding the wire device to the positive-connection element.

According to a further development of the present invention, after the material bonding, the wire device is materially bonded to the pushed-on sleeve on the central portion, so that the electrical conductor is received within the sleeve.

In some embodiments of the invention, the step of providing the rod may comprise a step of creating the local clearance in the rod, in particular by molding, e.g., pressing, or by machining—for example, milling or turning.

Optionally, the end and if necessary the contact portion can be provided with a capillary adhesive after the material bonding. For example, the end and optionally the contact portion can be dipped into a capillary adhesive. Capillary action allows gaps to be filled with adhesive.

The above embodiments and developments can be combined with each other as desired, if appropriate. Further possible embodiments, developments, and implementations of the invention also include combinations, which are not explicitly mentioned, of features of the invention described above or below with respect to the exemplary embodiments. In particular, a person skilled in the art will also add individual aspects as improvements or additions to the particular basic form of the present invention.

The present invention is explained in greater detail below with reference to the exemplary embodiments shown in the schematic figures of the drawing. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is an exploded view of an embodiment of a force transmission element;

FIG. 2 is a schematic side view of the force transmission element of FIG. 1 in an assembled state;

FIG. 3 is an isometric view of a proximal end of the force transmission element;

FIG. 4 is a transparent isometric view of a sleeve of the force transmission element;

FIG. 5 is a side view of an embodiment of a hand-guided surgical instrument;

FIG. 6 is a longitudinal section through the hand-guided surgical instrument in the region of a bearing element of an actuation interface; and

FIG. 7 is a flowchart of an embodiment of the method for producing a force transmission element.

### DESCRIPTION OF EXEMPLARY EMBODIMENTS

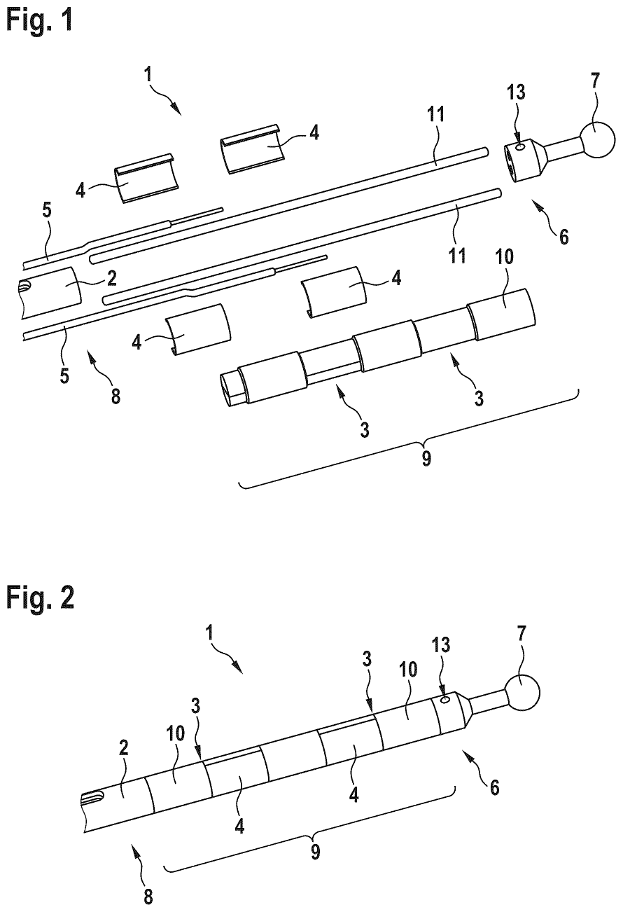

Referring to the drawings, in FIGS. 1 and 2, an embodiment of a force transmission element 1 is illustrated. In particular, FIG. 1 shows the force transmission element 1 in an exploded view.

The force transmission element 1 is designed in particular as a pull rod for an endoscopic instrument. The pull rod 1 comprises a rod 2, two circumferential clearances 3, two electrodes 4, two electrical conductors 5, a proximal end 6 with a positive-connection element 7, a sleeve 10 made of ceramic material, and a wire device 11.

The rod 2 is potential-free, designed for force transmission, and has two clearances 3 in a contact portion 9. The contact portion 9 has the sleeve 10 made of ceramic material. In particular, the sleeve 10 has the two circumferential clearances 3. Furthermore, the rod 2 comprises a central portion 8, wherein the contact portion 9 is arranged between the central portion 8 and the proximal end 6. The central portion 8 can be insulated from the two electrodes 4 by the sleeve 10.

For example, the two clearances 3 have a substantially flat portion, parallel to the axial direction of the rod 2, which is located further inwards in the radial direction compared to an outer surface of the rod and is in contact with the respective electrode 4 along a flat surface.

The wire device 11 contains, for example, two traction wires. These traction wires 11 each extend in the axial direction along the rod 2 through the contact portion 9 and are designed to transmit force between the central portion 8 and the proximal end 6. The two traction wires 11 are surrounded by the sleeve 10. Both traction wires 11 are connected to the positive-connection element 7 and the central portion 8 of the rod 2 by means of a welded connection. The sleeve 10 is positively secured by the wire device 11 between the positive-connection element 7 and the central portion 8. The proximal end 6 and the central portion 8 each have two receiving regions for receiving the ends of the two traction wires 11. For example, the two receiving regions are designed as blind-holes. Furthermore, the proximal end 6 contains a recess 13 which extends in the radial direction and crosses at least one of the blind-holes.

The sleeve 10 here contains four bores which extend substantially in the axial direction, wherein two of the four bores are designed to receive the two electrical conductors 5, and the other two of the four bores are designed to receive the two traction wires 11. In the area of the clearance 3, the sleeve 10 contains a recess which connects the bore for the electrical conductor 5 with an outer surface of the clearance 3, so that the electrical conductor 5 can protrude from the sleeve 10 through the recess. The sleeve 10 is manufactured in one piece using a generative manufacturing process.

The two electrical conductors 5 can, for example, each have an electrically insulating sheath or coating.

FIG. 2 shows a schematic side view of the force transmission element in an assembled state.

The two electrodes 4 extend over the outer surface of the clearance 3 and at least partially in the axial direction along the clearance 3. The electrodes 4 are each in contact with one of the two electrical conductors 5, which extends within the rod 2, in particular in the bore of the sleeve 10 provided for this purpose. The two electrodes 4 each have two half-shells which together surround the rod 2 circumferentially in the clearance 3. This means that the electrode 4 is divided into two half-shells, which together form a ring. The electrode 4 lies circularly and concentrically on the rod 2 in the region of the clearance 3.

The positive-connection element 7 of the proximal end 6 is electrically insulated. For example, the positive-connection element 7 is spherical and made of a metal. The spherical positive-connection element 7 serves to engage with a bearing element 103 or operating element of a surgical instrument 100. The positive-connection element 7 protrudes from the rod 2 in the axial direction. A diameter of the positive-connection element 7 is smaller than a diameter of the rod 2.

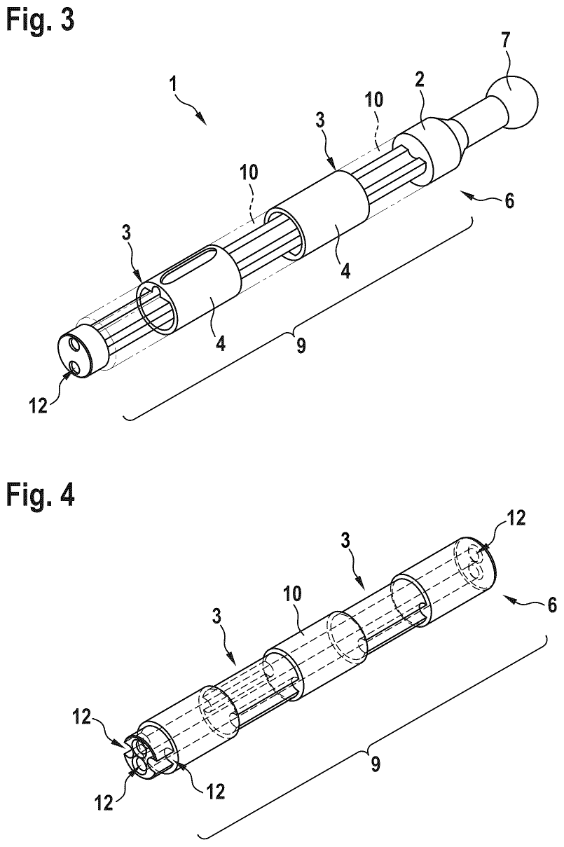

FIG. 3 shows an isometric view of a proximal end 6 of the force transmission element 1. The force transmission element 1 shown here comprises essentially the same features as the embodiment according to FIGS. 1 and 2, unless otherwise stated.

The sleeve 10 is illustrated transparently, to depict the interior of the contact portion 9 in an assembled state.

The clearance 3 in the rod 2 is circular and has a distal stop of the clearance 3 and a proximal stop of the clearance 3. The two electrodes 4 lie circularly and concentrically on the rod 2 in the region of the respective clearance 3.

On one end face of the contact portion 9, in particular on its distal end face, the two bores 12 for the two electrical conductors 5 can be seen.

FIG. 4 shows a transparent isometric view of a sleeve 10 of the force transmission element 1. In particular, the complex internal structure of the sleeve 10 with four internal bores 12 for guiding the two traction wires and the two electrical conductors 5 or cables is shown.

The sleeve 10 is, for example, made in one piece from a ceramic material by a generative manufacturing process. In the one-piece production, two circumferential clearances 3 are already taken into account, which are arranged at a distance from each other in the axial direction.

Two of the four internal bores 12 are designed as through-bores for the two traction wires in the axial direction. The other two of the four internal bores 12 are provided for the two electrical conductors and also extend in the axial direction, wherein these bores each end/begin in the region of the clearance 3. For example, the two bores 12 for the electrical conductors extend from the clearance 3, not in the direction of a proximal end 6, but in the direction of a distal end of the force transmission element or the pull rod 1.

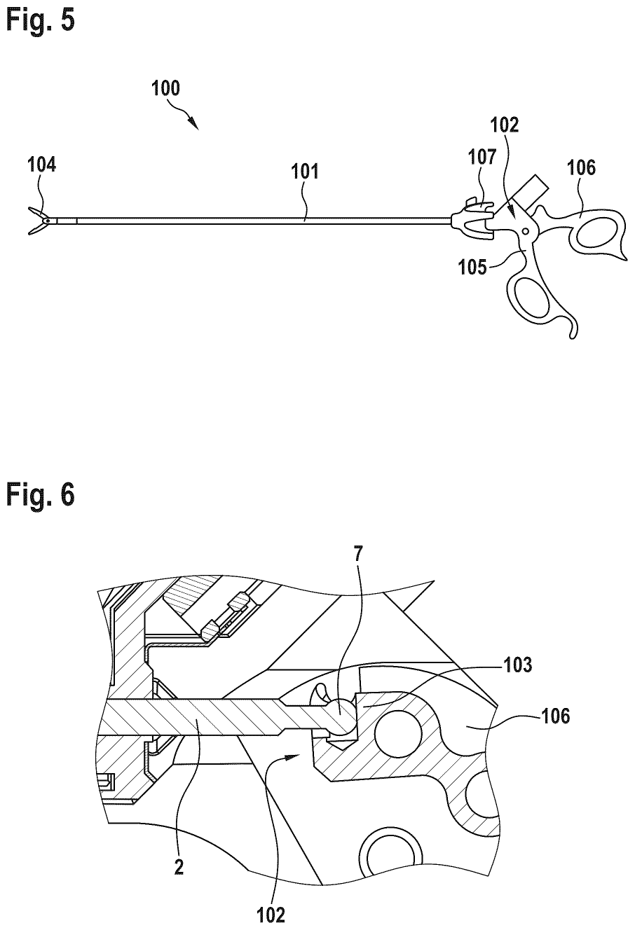

FIG. 5 is a side view of an embodiment of a hand-guided surgical instrument 100. The surgical instrument 100 comprises a force transmission element (not shown here; see FIGS. 1 to 4), a tubular shaft 101, an actuation interface 102, a bearing element (not shown here; see FIG. 6), a tool or accessory 104, a grip 105, a movable grip limb 106, and an accessory interface 107.

The force transmission element is accommodated and mounted in the tubular shaft 101. The bearing element secures the force transmission element against axial displacement relative to the tubular shaft 101 (cf. FIG. 6). The tool 104 is designed here as a gripper, which can be supplied with both poles of the electrical current via the force transmission element. The surgical instrument 100 is equipped with a grip 105 for manual guidance by a user (e.g., surgeon). Alternatively, the surgical instrument 100 may, for guidance by a robot, be equipped with a corresponding connection interface for the robot (not shown). The movable grip limb 106 is used for manual force application. The force applied to the grip limb 106 is transmitted through the grip limb 106 to the force transmission element. The force transmission element in turn transmits the axial force to the tool/accessory. In addition, two electrical poles of a bipolar generator (not shown) are connected to the tool 104 via the force transmission element. The tool/accessory 104 can be detachably mechanically connected to the grip 105 via the accessory interface 107.

FIG. 6 shows a longitudinal section through the hand-guided surgical instrument 100 in the region of a bearing element 103 of an actuation interface 102.

The bearing element 103 engages a spherical positive-connection element 7 of a force transmission element. In addition, a rod 2 of the force transmission element is shown, which is coupled with its proximal end to the actuation interface 102 for mechanical actuation. By lever-like manipulation of the movable grip limb 106, the rod 2 is displaced axially relative to the surgical instrument. The bearing element 103 is designed, for example, like a fork, so that two limbs of the fork-like bearing element 103 can positively engage the spherical positive-connection element and, in particular, can pull the rod 2. In addition, the bearing element 103 can axially press the spherical positive-connection element.

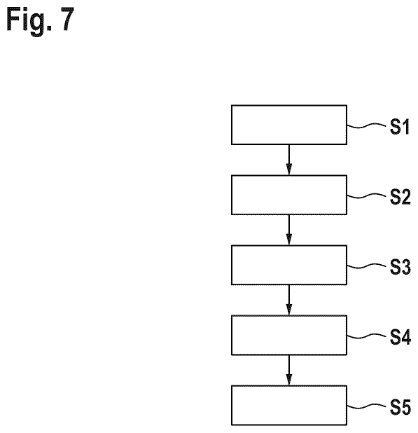

FIG. 7 schematically shows an embodiment of the method for producing the force transmission element for surgical instruments, in particular a force transmission element 1 of FIGS. 1 to 4. The method comprises the steps of providing S1, pushing-through S2, materially bonding S3, materially bonding S4, and applying S5.

In the providing step S1, an insulating rod 2 designed for force transmission is provided, which has at least one local clearance 3 and one end 6, in particular a proximal end, with an electrically insulated positive-connection element 7. The positive-connection element 7 is designed to engage with a bearing element 103 of a surgical instrument 100. The rod 2 has a central portion 8 and a contact portion 9, arranged between the central portion 8 and the end 6, in which the at least one clearance 3 is arranged.

In the pushing-through step S2, a wire device 11 is pushed through a sleeve 10, made of ceramic material, provided in the contact portion 9. The wire device 11 contains, for example, multiple traction wires. The traction wires 11 are positioned by the sleeve 10. The traction wires are inserted into the proximal end or end piece of the rod until they stop. In addition, approximately the rearward ⅔ of the traction wires can be wetted with an adhesive before the sleeve is pushed on.

Step S3 comprises the material bonding of the traction wires 11 with the positive-connection element 7. The traction wires 11 are welded at the front and through a recess 13 of the proximal end, which extends in the radial direction. One end face of the proximal end can be fully wetted with an adhesive and pressed with the sleeve. After welding, the material bonds can be cured at around 100° C. for about 15 minutes.

After the contact portion is materially bonded to the proximal end S3, the traction wires 11 are materially bonded to the central portion 8 with the pushed-on sleeve 10 in step S4, so that the electrical conductor 5 is received within the sleeve 10. The electrical conductor is threaded through the sleeve. The electrical conductor can be approximately 6-10 cm longer than is necessary when installed. Consequently, the electrical conductor is cut to a suitable length only once it has been assembled. The sleeve is glued into the central portion, for example. The traction wires are welded to the central portion using an additive. Preferably, the temperature development at the welding point is observed in order to avoid damage to the electrical conductor during the welding process, due to the very narrow cable routing. For example, welding is carried out only along the traction wires. The weld site can additionally be provided with a temperature-resistant adhesive and/or a filler. Following welding and gluing, the material bonds can be cured at around 100° C. for about 15 minutes.

In the application step S5, an electrode 4 is applied, which extends over an outer surface of the clearance 3 and at least partially in the axial direction along the clearance 3. The electrode 4 is brought into contact with the electrical conductor 5, which extends inside the rod 2. The application S5 of the electrode 4 can comprise stripping and welding, soldering, and/or crimping the electrical conductor 5 to the electrode. The electrode can, for example, consist of two electrode half-shells. In particular, the electrical conductor is welded to an inner side of the electrode half-shell.

Optionally, the method may include a step of testing the electrical connection/insulation. A contact resistance is measured from the electrode half-shell up to an electrode in the tool of a surgical instrument. In addition, the resistance of the electrode half-shell to the other tool as well as the resistance of the electrodes with respect to the force transmission element or the pull rod are measured.

After applying S5 the electrode half-shells, a weld seam, optionally with additional material, can be created to connect the electrode half-shells. All welds are ground. It should be noted that the electrode half-shell is secured against rotation only by the weld on the electrical conductor.

Furthermore, a capillary adhesive can optionally be applied. For example, the proximal end is dipped into the capillary adhesive so that gaps between the electrode half-shells and the sleeve or a cable duct are filled by the capillary action.

In the foregoing, detailed description, various features have been summarized in one or more examples to improve the stringency of the presentation. It should be understood, however, that the above description is merely illustrative and not restrictive. It is intended to cover all alternatives, modifications, and equivalents of the various features and exemplary embodiments. Many other examples will be immediately and directly clear to a person skilled in the art on the basis of their technical knowledge in view of the above description.

The exemplary embodiments were selected and described in order to best illustrate the principles underlying the invention and its possible applications in practice. This allows for those skilled in the art to optimally modify and utilize the invention and its various exemplary embodiments in relation to the intended purpose. In the claims and the description, the terms “including” and “having” are used as neutral language terms for the corresponding terms “comprising.” Furthermore, the use of the terms “a” and “an” is not intended to exclude a plurality of features and components described in this way.

While specific embodiments of the invention have been shown and described in detail to illustrate the application of the principles of the invention, it will be understood that the invention may be embodied otherwise without departing from such principles.

### LIST OF REFERENCE SIGNS

1 force transmission element
2 rod
3 clearance
4 electrode
5 electrical conductor
6 end
7 positive-connection element
8 central portion
9 contact portion
10 sleeve
11 wire device
12 bore
13 recess
100 surgical instrument
101 tubular shaft
102 actuation interface
103 bearing element
104 tool/accessory
105 grip
106 moving grip limb
107 accessory interface
S1 providing
S2 pushing-through
S3 material bonding
S4 material bonding
S5 applying

What is claimed is:

1. A force transmission element for surgical instruments, the force transmission element comprising:
a potential-free rod configured for force transmission, the rod having at least one local clearance; and
an electrode which extends over an outer surface of the clearance and extends at least partially in the axial direction along the clearance, wherein the electrode is in contact with an electrical conductor which extends within the rod; wherein one end of the rod has an electrically insulated positive-connection element which is configured to engage with a bearing element of a surgical instrument.

2. The force transmission element according to claim 1, wherein the rod comprises a central portion and a contact portion, the contact portion being arranged between the central portion and the end, in which contact portion the at least one clearance is arranged, wherein the contact portion comprises a sleeve made of ceramic material.

3. The force transmission element according to claim 2, wherein the contact portion comprises a wire device for force transmission between the central portion and the end, wherein the wire device extends in the axial direction along the rod and is at least partially surrounded by the sleeve.

4. The force transmission element according to claim 3, wherein the wire device is connected to the positive-connection element and the central portion of the rod with a material bond, wherein the sleeve is fastened at least with a positive connection between the positive-connection element and the central portion by the wire device.

5. The force transmission element according to claim 4, wherein the wire device is connected to the positive-connection element and the central portion with a material bond.

6. The force transmission element according to claim 5, wherein the material bond comprises a welded connection or a capillary adhesive.

7. The force transmission element according to claim 1, wherein the sleeve is manufactured in one piece by a generative manufacturing process or by injection molding.

8. The force transmission element according to claim 1, wherein the clearance has at least one substantially flat portion, parallel to the axial direction of the rod, which is located further inwards in the radial direction compared to an outer surface of the rod and is in contact with the electrode by lying against it.

9. The force transmission element according to claim 8, wherein the clearance lies flat against the electrode.

10. The force transmission element according to claim 1, wherein the positive-connection element is spherical.

11. A surgical instrument comprising:
a force transmission element comprising: a potential-free rod configured for force transmission, which has at least one local clearance, and an electrode which extends over an outer surface of the clearance and at least partially in the axial direction along the clearance, wherein the electrode is in contact with an electrical conductor which extends within the rod, wherein one end of the rod has an electrically insulated positive-connection element which is configured to engage with a bearing element of a surgical instrument;
a tubular shaft in which the force transmission element is received;
an actuation interface configured to actuate the force transmission element;
a bearing element which is integrated into the actuation interface and which couples the positive-connection element of the force transmission element; and
a tool movable by means of the force transmission element.

12. The surgical instrument according to claim 11, wherein the tubular shaft is configured as a round tube, wherein the rod of the force transmission element is configured as a round rod and the sleeve extends at least partially circularly along the surface of the rod, and wherein the force transmission element is arranged concentrically in the tubular shaft.

13. A method for producing a force transmission element for surgical instruments, the force transmission element comprising a potential-free rod configured for force transmission, which has at least one local clearance, and an electrode which extends over an outer surface of the clearance and at least partially in the axial direction along the clearance, wherein the electrode is in contact with an electrical conductor which extends within the rod, wherein one end of the rod has an electrically insulated positive-connection element which is configured to engage with a bearing element of a surgical instrument, the method comprising the following steps:
providing the potential-free rod configured for force transmission, the potential-free rod having at least one local clearance and one end with the electrically insulated positive-connection element configured to engage with a bearing element of a surgical instrument; and
applying an electrode which extends over an outer surface of the clearance and at least partially in the axial direction along the clearance, wherein the electrode is brought into contact with the electrical conductor which extends within the rod.

14. A method according to claim 13, wherein the step of applying the electrode comprises stripping and welding, soldering, and/or crimping the electrical conductor to the electrode.

15. A method according to claim 13, wherein the rod has a central portion and a contact portion arranged between the central portion and the end, in which contact portion the at least one clearance is arranged, wherein a wire device is pushed through a sleeve, made of ceramic material, provided in the contact portion.

16. A method according to claim 13, wherein the method further comprises the step of materially bonding the wire device to the positive-connection element.

17. A method according to claim 14, wherein after the material bonding, the wire device is materially bonded to the pushed-on sleeve at the central portion so that the electrical conductor is received within the sleeve.
